(19) United States
(12) Patent Application Publication
(10) Pub. No.: US 2005/0241645 A1
(43) Pub. Date: Nov. 3, 2005

(54) Composition for preventing the transmission of human immunodeficiency syndrome virus

(76) Inventors: Joseph Vernice, Shirley, NY (US); Alfred R. Globus, Hauppauge, NY (US)

Correspondence Address: Evelyn M. Sommer, Suite 825, 250 Park Avenue, New York, NY, 10022, US

(21) Appl. No.: 11/100,294

(22) Filed: Apr. 6, 2005

Related U.S. Application Data
(60) Provisional application No. 60/560,218, filed on Apr. 8, 2004.

(51) Int. Cl. A61F 6/06

(52) U.S. Cl. 128/832

(57) ABSTRACT

A gel composition and methods for its use singly or in combination with prophylactic devices in safe sexual relations, including prevention of infection by HIV and other sexually transmitted viruses. The composition contains benzoic acid as the active ingredient in an amount up to 2%, a copolymer of polyquaternium-4 and polyacrylamidomethylpropane sulfonic acid and a pharmaceutically acceptable carrier such as a water base consisting of about 60% water and about 20% glycerin. The composition may further contain a preservative, such as one or more members of parabens. The gel can maintain its antiviral activity in dilutions of not less than 1000 times. The methods for preventing the transmission of HIV and other sexually transmitted viruses comprise applying the gel composition directly onto genitalia or intravaginally before and/or during sexual intercourse or using the gel composition in combination with a prophylactic device, such as a condom or a diaphragm.

This application claims priority of Provisional Application Ser. No. 60/560218 filed Apr. 18, 2004.

### FIELD OF THE INVENTION

The present invention relates to a gel composition for use in reducing the risk of infection caused by sexually transmitted viruses, such as herpesviruses (herpes simplex, cytomegalovirus and the like), influenza A, parainfluenza, hepatitis B, human papilloma viruses, and in particular human immunodeficiency virus (HIV). The present invention also relates to a method for reducing the risk of infection by the above identified viruses as a result of sexual intercourse by the use of the gel composition during sexual intercourse.

### BACKGROUND OF THE INVENTION

Acquired human immunodeficiency syndrome (commonly abbreviated as AIDS) is a disease caused by human T-cells becoming infected by a causative virus present in human blood. The virus is known as acquired human immunodeficiency syndrome virus which commonly is abbreviated as HIV.

The spread of HIV worldwide has caused significant loss in both human lives and in the economy, especially in third world countries such as sub-Saharan Africa and, increasingly, in some Asian countries. The most promising drugs developed and currently under development include, for instance, azidothymidine (AZT), bis-deoxycytidine, suramine, phosphonoformate, antimony tungstate, papaverine as disclosed in U.S. Pat. No. 4,971,977, 2′,3′-dideoxyinosine as disclosed in U.S. Pat. No. 5,026,687, benanomicins A and B as disclosed in U.S. Pat. No. 5,120,717, porphyrin as disclosed in U.S. Pat. No. 5,192,788, and inhibitory peptides as disclosed in U.S. Pat. No. 4,880,779. One major limitation of these drugs and many others is that they are typically highly toxic. Another important limitation is that these drugs are very expensive and therefore are not affordable by the economically disadvantaged patients in the developing countries where HIV infections strike hardest. One further limitation is that the regimen for their use is typically tedious and hard to follow. However non-adherence to the regimen can compromise the efficiency of the drug and also enhance the possibility of resistance to the drug by the virus.

It has been concluded that the most effective way to control the spread of HIV infection and AIDS disease is to prevent the infection of a healthy person by that virus.

HIV can be transmitted from an infected person to a healthy person when the healthy person comes into contact with the blood, semen, vaginal fluids, and breast milk from the infected person. The four general modes of HW transmission include: unprotected sex, blood contact, the use of HIV virus contaminated syringes and/or needles, and mother to child before, during and after childbirth. A major method of HIV transmission could take place during unprotected sexual intercourse due to the exchange of bodily fluid. Therefore, AIDS is regarded as a venereal disease with a fatal outcome, and in as many as 80% of the world's diagnosed cases, is caused by the passage of HIV across the genital mucosa (U.S. Pat. No. 5,552,383 and Merson, Science, 260: 1266-8, 1993). While condoms can be used to reduce the likelihood of transmission of HIV virus during sexual contacts, the use of condoms is not always strictly adhered to due to various reasons, including their cost, religious prohibition, and the diminished sexual satisfaction.

A method which is convenient and easily implemented, for reducing the risk of contracting HIV and the other sexually transmitted viruses, during sexual intercourse is by the use of a pharmaceutical composition, in the form of gel, lubricant cream or the like, by at least one of the individuals involved. Clinical surveys have suggested that the use of spermicides with known antiviral activity, e.g. nonoxynol-9, will not satisfactorily restrict the spread of HIV (Cates et al. Family Planning Perspective, 24:75-84, 1992), as the frequent use of nonoxynol-9 leads to the creation of vaginal lesions (genital ulcers and vulvitis), which provide entry ports for HIV particles to pass through the protective barrier of the skin and reach the bloodstream (Kreiss et al. The Journal of The American Medical Association, 268: 477-482, 1992). Kelly in U.S. Pat. No. 5,624,675 disclosed a method of using a topical genital lubricant with a non-toxic, non-irritating zinc salt during sexual intercourse for the reduction of HIV infection. The mechanisms are such that 1) zinc can promote healing and closure of lesions, microabrasions, and other skin breaches and thereby reduces the ability of HIV to penetrate the skin and reach lymphocytes, 2) zinc ions form crosslinking bonds with cysteine and histidine residues in the HIV protein and thereby attach HIV particles to each other, to proteins in vaginal fluids, and to dead or dying cells that will be soon discharged from the vaginal surface which reduces the ability of the HIV virus to infect the susceptible healthy cells, and 3) concentrations of zinc that do not harm the skin can kill HIV-infected lymphocytes and prevent the lymphocytes from further infecting other cells. Bourinbaiar in U.S. Pat. No. 5,552,382 disclosed the use of gramicidin as an active ingredient of spermicide with virucidal activity against HIV. Shihata in U.S. Pat. No. 5,778,886 disclosed the use of nonoxynol-9 and hydrogen peroxide in vaginal compositions for the prevention of conception and the transmission of sexually transmitted disease. Porat in U.S. Pat. No. 6,624,198 disclosed a spermicidal lubricant composition, which includes chlorhexidine salt as an active ingredient against HIV and other viruses.

### SUMMARY OF THE INVENTION

In accordance with the invention it has now been found that the use of a specially formulated gel as a lubricant during sexual intercourse, which contains, as an active ingredient, benzoic acid in a suitable pharmaceutical carrier, can significantly de-vitalize the HIV virus, and/or other sexually transmitted disease viruses upon contact within a reasonable period of time. The gel is also capable of serving as a contraceptive by devitalizing the sperm.

It is a primary object of this invention to provide a composition for the prevention of the transmission of HIV and other sexually transmitted disease viruses during sexual intercourse.

It is a primary object of this invention to provide a method for the prevention of the transmission of HIV and other sexually transmitted disease viruses during sexual intercourse.

It is another object of this invention to provide a method for utilizing benzoic acid in a suitable formulation that can be spread on the genitals prior to or during sexual intercourse to reduce the risk that uninfected person will become infected by HIV and other viruses.

Another object of this invention is to provide a sexual lubricant, gel, or cream which contains an effective topically-active anti-HIV agent which is non-toxic and non-irritating to the genitals and urethral and vaginal membranes.

The compositions can be applied to the external genitalia as well as internal mucosal surface to prevent transmission of HIV and other sexually transmitted disease viruses.

Yet another object of this invention is to provide a sexual lubricant, gel, or cream which contains an effective topically-active anti-HIV agent, which is non-toxic and non-irritating to the genitals and urethral and vaginal membranes and which can be used in conjunction with a prophylactic device, such as a condom or a diaphragm.

### DETAILED DESCRIPTION OF THE INVENTION

In order to maintain the activity of benzoic acid against HIV and other sexually transmitted disease viruses, it is essential to maintain the pH value of the composition at or below 4.0. The pH of a healthy vagina is also mildly acidic (pH 3.5-4.5) and this acidity is generated by the production of lactic acid by lactobacilli, which form a major component of the healthy vaginal flora. Together with other factors, the acid pH is recognized as an essential part of the protective mechanisms against infections. While the acidic conditions can inactivate sperms and viruses, semen contains a potent alkaline buffering capacity that neutralizes the vaginal acidity for a period of many hours after intercourse, therefore diminishing the virus inhibiting capacity of the acids (Lezdey et al in U.S. Pat. No. 6,428,791).

Regular gels do not perform well under such acidic conditions because the acidity tends to destabilize the gels. In accordance with the invention a special gel is provided, “Origen”, that can be used in acidic conditions as exist in the vagina and surrounding area without destabilization. The gel is made by the co-polymerization of polyquaternium-4 and polyacrylamidomethylpropane sulfonic acid in a pharmaceutically acceptable carrier. A preferred carrier for this purpose is a water base consisting about 60% water and about 20% glycerin. The presence of the glycerin is important in that it increases the spread of the gel on the surface of genitals. The lubricity of the gel further helps reduce the likelihood of transmitting the diseases by reducing the rupture of skins and/or blood vessels during sexual relationships. The gel of the invention is activated by being made alkaline with sodium hydroxide to produce a uniform co-polymerization and solubilization, then acidified with hydrochloric acid to reach the desire acid pH of about 3.8.

Polyquaternium-4, commercially available from companies such as National Starch under the trade name CELQUAT® L-200 or H-100, has been used primarily in the skin and hair care industry in formulations in conditioners, cream rinses, creams, detangling spritzes, liquid soaps, mousses, setting lotions and skin lotions. Polyquaternium-4 is a cationic, polymeric, and water soluble quaternary cellulose derivative have the following structure.

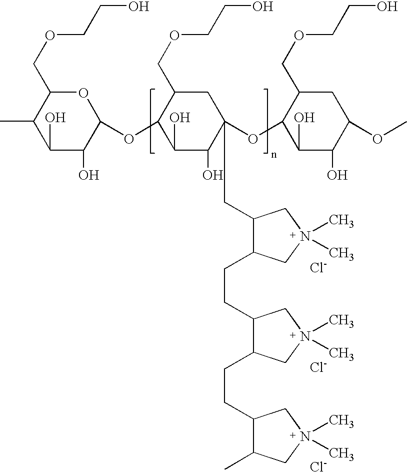

Polyacrylamidomethylpropane sulfonic acid, whose structure is shown below, has theretofore been used as a film former, a thickener, a lubricity additive and a moisturizer for personal care and home care applications (U.S. Pat. No. 4,128,631). It is commercially available, for example, under the trade name COSMEDIA® HSP-1180 by Cognis.

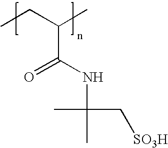

To the gel with the co-polymerized polyquaternium-4 and polyacrylamidomethylpropane sulfonic acid in the above-mentioned water base, a preservative can be added. A preferred preservative is selected from one or more type of parabens. The parabens are a group of closely related chemicals which are esters of p-hydroxybenzoic acid. They are used widely as preservatives for cosmetics, foods and drugs. Other known preservatives or combinations thereof can also be used instead, especially for the formulations prepared for the individuals that are allergic to parabens. The active, benzoic acid should be added at a concentration generally of about 0.2% to no more than 2%, as too great an amount will inhibit the normal and necessary flora in the vaginal tract. The benzoic acid further acts as a stabilizer to prevent a fall off of activity if the product should become diluted.

The following preferred formulation of the gel of this invention is provided as an example,

| Ingredient | Amount (% by wt) |
|---|---|
| Polyquaternium-4 | 0.70 |
| Polyacrylamidomethylpropane | 3.30 |
| sulfonic acid | |
| Water | QS to 100% |
| Glycerin | 10.00 |
| Sodium hydroxide | 0.15 |
| Hydrochloric acid | QS to pH 3.8 |
| Benzoic acid | 0.30 |
| Methylparaben | 0.20 |

In order to evaluate the efficacy of the Origen gel in inhibiting HIV viruses in both concentrated and diluted forms, the following tests were conducted.

Four sets of samples were prepared according to the following steps which are test samples based on Origen gel at a first series of dilutions, control samples based on AZT at a second series of dilutions, a positive control sample and a negative control sample.

Step 1. For the Origen samples, Origen gel was diluted in sterile distilled water giving rise to dilutions ranging from 1:5 to 1:500,000 (2× concentrations). For the AZT samples, an AZT stock was diluted in H9 media giving rise to dilutions whose concentrations were 2, 0.2 and 0.02 μM (2× concentrations). The H9 cells are a type of HIV-1 permissive human T-cell lymphoma. Typical H9 media comprise RPMI, fetal bovine serum (FBS), penicillin, streptomycin and glutamine, for example, RPMI 1640 supplemented with 20% fetal bovine serum, 2 mm 1-glutamine, 100 U·mL⁻¹ penicillin, and 100 μg·mL⁻¹ streptomycin, and 5% CO₂.

Step 2. For each of the Origen samples, an aliquot of 50 μl Origen gel dilution was mixed with 50 μl HIV-1 (500 TCID₅₀) in a 1.2 milliliter centrifuge tube by vortexing to provide final Origen gel dilutions (1:10 to 1:1,000,000). For each of the AZT samples and the positive control sample, 50 μl water was mixed with 50 μl HIV-1 in a 1.2 milliliter centrifuge tube by vortexing. For the negative control sample, 100 μl of water was added to a 1.2 milliliter centrifuge tube.

Step 3. The sample tubes were incubated for 15 minutes at 37° C. and 5% humidity.

Step 4. An aliquot of 900 μl media for H9 cell was added to each tube. The tubes were centrifuged at 17,000 rpm at 4° C. for 90 minutes to pellet the viruses. The supernatants were discarded.

Step 5. For the Origen samples and the positive control sample, each pellet was re-suspended in 500 μl H9 media by vortexing. For the AZT samples, each pellet was re-suspended in 500 μl of 2× AZT dilution by vortexing.

Step 6. An aliquot of 100 μl sample was transferred from each tube into quadruplicate wells in a 96-well flat-bottomed plate.

Step 7. An aliquot of 100 μl H9 cells (diluted to 1×10⁶ cells/ml in H9 media) were added to each well, giving rise to a final concentration of H9 cells of 5×10⁵/ml. The AZT control samples have final concentrations of 1, 0.1, 0.01 50 μM.

Step 8. The samples were incubated for 3 days at 37° C. and 5% humidity.

Step 9. On the beginning of the fourth day, the culture supernatant was removed and discarded. For the Origen samples, the positive control sample and the negative control sample, 100 μl fresh H9 media were added. For the AZT samples, 100 μl 1× AZT dilution was added.

Step 10. The samples were further incubated for 3 more days at 37° C. and 5% humidity.

Step 11. On the beginning of the seventh day, the H9 cells from the culture were collected for indirect immunofluorescence assay (IFA) to detect HIV-1 antibodies.

Step 12. Viability of the H9 cells was checked using trypan blue exclusion method.

The result of the test is summarized in the following table.

| Drug dilution | % cell viability | % IFA | % inhibition |
|---|---|---|---|
| Origen Gel 1:10 | 80% | 0% | 100% |
| Origen Gel 1:100 | 80% | 0% | 100% |
| Origen Gel 1:1000 | 80% | 0% | 100% |
| AZT 1 μM | 80% | 0% | 100% |
| AZT 0.1 μM | 80% | 20% | 77.8% |
| AZT 0.01 μM | 80% | 80% | 11.2% |
| Positive control | 80% | 90% | NA |
| Negative control | 90% | 0% | NA |

The results show that Origen gel is generally nontoxic and that it can completely inhibit the HIV viruses at dilutions as high as 1000 times. Therefore, the gel comprising the above-mentioned compositions is capable of destroying any HIV and/or other sexually transmitted viruses that come into contact therewith in the vaginal tract in a matter of hours to a maximum of one day. Due to the ability of benzoic acid for deactivating sperm, the gel provides a further advantage by serving as a contraceptive. The composition further possesses the following advantageous properties, 1) the composition is compatible with the conditions necessary to maintain a healthy vaginal condition, 2) the composition is of excellent stability and freedom from self-liquefaction in that body cavity that might cause the composition to run or leak from he cavity; 3) the composition continues to be effective even if significantly diluted by some bodily fluids; and 4) the composition can be produced for sale at costs low enough to be obtainable and usable by the affected people of the world, especially those with limited incomes.

The gel can be directly applied to either the male or female genitalia (such as penis and/or vagina) or any other bodily cavities, such as the rectum, which might be involved in the sexual intercourse prior to the sexual intercourse. The gel can also be used as a lubricant in combination with a prophylactic device, such as a condom or a diaphragm. When a condom is used in conjunction with the gel, the gel is capable of enhancing the risk-reducing effectiveness of condom and providing maximum protection for users. Such an increase in the anti-viral protection offered by a condom becomes important in various situations, such as when a condom breaks or if the male loses his erection prior to withdrawal and spillage or leakage of fluid occurs from the inside of the condom into the vagina. The gel can be coated onto condoms during manufacture, and enclosed within conventional watertight plastic or foil packages that contain one condom per package, or it can be manually applied by a user to the inside and/or the outside of a condom, immediately before use. The Origen gel composition can also be used by doctors and in hospitals to lubricate and sanitize gloves or instruments before examining the penis, vagina, rectum and the like.

During sexual intercourse, the active in the gel, benzoic acid, deactivates the viruses in a matter of several hours to a maximum of one day and prevents the infected person from transmitting sexually transmitted disease viruses to the non-infected person. The ability of the gel to maintain its efficacy up to 1000× dilution is of paramount importance for the intended protection due to the secretion of bodily fluids inside the bodily cavities which dilutes the gel during sexual intercourse. The gel provides lubricity which further helps reduce the likelihood of transmitting the diseases by reducing the rupture of skins and/or blood vessels during sexual relationships.

The Origen gel can be packaged in any of the known forms, such in tubes, in bottles, in capsules or as an impregnant for treated towlettes which can be used to wipe the sex organs.

1. A gel composition for topical application to at least one of the sex organs selected from the group consisting of penis, vagina and rectum of the individuals involved in a sexual activity for preventing transmission of sexually transmitted diseases viruses, comprising a copolymer of polyquaternium-4 and polyacrylamidomethylpropane sulfonic acid and benzoic acid in a pharmaceutically acceptable carrier.

2. The gel composition according to claim 1 wherein said sexually transmitted disease virus is selected from the group consisting of HIV viruses, herpesviruses, hepatitis viruses, influenza A viruses, parainfluenza viruses, and human papilloma viruses.

3. The gel composition according to claim 1 wherein said benzoic acid is present in an amount between about 0.2 and 2%.

4. The gel composition according to claim 1 wherein said carrier is a water base consisting of about 60% water and about 20% glycerin.

5. The gel composition according to claim 1 wherein said gel further comprises a preservative.

6. The gel composition according to claim 5 wherein said preservative is selected from the group consisting of parabens and mixtures thereof.

7. A method of preventing the transmission of sexually transmitted disease viruses which comprises applying the gel composition according to claim 1 onto said sex organs before sexual intercourse.

8. A method of preventing the transmission of sexually transmitted disease viruses which comprise applying the gel composition according to claim 1 onto a prophylactic device before using said device during sexual intercourse.

9. A method of preventing the transmission of sexually transmitted disease viruses according to claim 8 wherein said prophylactic device is a condom.

10. A method of preventing the transmission of sexually transmitted disease viruses according to claim 8 wherein said prophylactic device is a diaphragm.
